(19) United States
(12) Patent Application Publication
(10) Pub. No.: US 2026/0328783 A1
(43) Pub. Date: Oct. 22, 2026

(54) BIOMARKER COMPOSITION FOR DIAGNOSIS OF DEGENERATIVE BRAIN DISEASE

(71) Applicant: JHK Medical Science Inc., Daejeon (KR)

(72) Inventor: Seo Yun JO, Daejeon (KR)

(73) Assignee: JHK Medical Science Inc., Daejeon (KR)

(21) Appl. No.: 19/176,244

(22) Filed: Apr. 11, 2025

(51) Int. Cl. G01N 33/68; C12Q 1/44; C12Q 1/527; C12Q 1/533

(52) U.S. Cl. CPC G01N 33/6896; C12Q 1/44; C12Q 1/527; C12Q 1/533; G01N 33/6872; G01N 2333/4703; G01N 2333/4709; G01N 2333/916; G01N 2333/988; G01N 2333/99; G01N 2800/2821

(57) ABSTRACT

The present disclosure relates to a biomarker composition for diagnosis of a degenerative brain disease, and more particularly, in an attempt to distinguish between normal people, mild cognitive impairment (MCI) patients, and Alzheimer's disease (AD) patients, provides a biomarker composition that distinguishes between the normal people, mild cognitive impairment (MCI) patients, and Alzheimer's disease (AD) patients by measuring levels of amyloid beta 40 (Aβ40), amyloid beta 42 (Aβ42), brain-derived neurotrophic factor (BDNF), Tau, neuron-specific enolase (NSE), glial fibrillary acidic protein (GFAP), ubiquitin carboxy-terminal hydrolase L1 (UCH-L1), peptidyl-prolyl isomerase (PIN1), collapsing response mediator protein-2 (CRMP2), phospho-Tau231, and olfactory marker protein (OMP) as biomarkers in the blood to identify correlation thereof.

### BACKGROUND

### 1.Field of the Invention

The present disclosure relates to a biomarker composition for diagnosis of a degenerative brain disease.

### 2. Acknowledgement

This work was supported by the Substantiation support program, through the Korea Innovation Foundation funded by the Ministry of Science and ICT.

### 3. Description of the Related Art

A degenerative brain disease refers to a disease that occurs in the brain among other degenerative diseases that develop with age and can be classified based on the main symptoms and the affected area in the brain, including, for example, Alzheimer's disease, frontotemporal dementia, Lewy body dementia, Parkinson's disease, Huntington's disease, and amyotrophic lateral sclerosis. Though the degenerative brain disease is known to be caused by neurodegeneration due to aging as well as death of nerve cells by aggregation of disease-specific proteins due to environmental factors, the exact pathological mechanism has not yet been identified, and thus the basic research has been actively being conducted to elucidate the mechanism.

Alzheimer's disease is a disease that develops gradually with memory loss and progresses to gradual, extensive loss of cognitive functions such as loss of spatial and temporal ability, language impairment, and executive function impairment, resulting in symptoms of dementia that make it impossible to perform independent daily life. Although the pathological mechanism has not yet been precisely identified, it is reported to be caused by the interaction among aging, genetic risk factors, and environmental factors.

According to the analysis results of dementia-related organizations such as the Ministry of Health and Welfare, National Health Insurance Service, Health Insurance Review & Assessment Service, Social Security Information Service, and Statistics Korea, the number of dementia patients at the end of 2017 is estimated to be over 700,000, the annual management cost per dementia patient is approximately 20.74 million won, and the national dementia management cost is approximately 14.6 trillion won. Currently, the prevention, diagnosis, and management of dementia are based on questionnaires such as clinical questionnaires or expensive examinations such as PET-CT, and since biological samples such as cerebrospinal fluid are required for diagnosis and management after the onset of the disease, patients are affected by the added burden of management costs. Moreover, as the population ages, the number of patients and costs are expected to increase consistently, and considering the additional loss of social costs, it is a huge burden on the country as well.

However, existing dementia diagnosis technology using blood has been conducted academically using a laser analyzer in units of pg/ [symbol] in serum or plasma, instead of using blood components. Conventional associated diagnostic kits utilize simple, low-sensitivity methods with limited markers that use the potential differences of a limited number of dementia markers, making it difficult to accurately diagnose and differentiate dementia. Therefore, in order to secure individuals' right to pursue health and reduce individual/national management costs, the need for an effective dementia diagnosis kit using blood is increasing in an attempt to prevent and detect dementia at an early stage and manage its progression.

### SUMMARY

### Problem to be Solved by the Invention

The present disclosure relates to a biomarker composition for diagnosis of a degenerative brain disease, and more particularly, in an attempt to distinguish between normal people, mild cognitive impairment (MCI) patients, and Alzheimer's disease (AD) patients, provides a biomarker composition that distinguishes between the normal people, mild cognitive impairment (MCI) patients, and Alzheimer's disease (AD) patients by measuring levels of amyloid beta 40 (Aβ40), amyloid beta 42 (Aβ42), brain-derived neurotrophic factor (BDNF), Tau, neuron-specific enolase (NSE), glial fibrillary acidic protein (GFAP), ubiquitin carboxy-terminal hydrolase L1 (UCH-L1), peptidyl-prolyl isomerase (PIN1), collapsing response mediator protein-2 (CRMP2), phospho-Tau231, and olfactory marker protein (OMP) as biomarkers in the blood to identify correlation thereof.

### Means for Solving the Problem

The present disclosure provides a diagnostic biomarker composition that distinguishes between normal people and mild cognitive impairment (MCI), including amyloid beta 40 (Aβ40), amyloid beta 42 (Aβ42), collapsing response mediator protein-2 (CRMP2), and phospho-Tau231 as active ingredients.

In addition, the present disclosure provides a diagnostic biomarker composition that distinguishes between normal people and Alzheimer's disease, including amyloid beta 40 (Aβ40), amyloid beta 42 (Aβ42), brain-derived neurotrophic factor (BDNF), phospho-Tau231, and olfactory marker protein (OMP) as active ingredients.

In addition, the present disclosure provides a diagnostic biomarker composition that distinguishes between mild cognitive impairment (MCI) and Alzheimer's disease, including amyloid beta 40 (Aβ40), amyloid beta 42 (Aβ42), Tau, glial fibrillary acidic protein (GFAP), and phospho-Tau231 as active ingredients.

In addition, the present disclosure provides a method of providing information for distinguishing between normal people and mild cognitive impairment (MCI), including measuring a level of a biomarker including amyloid beta 40 (Aβ40), amyloid beta 42 (Aβ42), collapsing response mediator protein-2 (CRMP2), and phospho-Tau231 from a biological sample isolated from a subject; measuring levels of one or more markers selected from Tau, ubiquitin carboxy-terminal hydrolase L1 (UCH-L1), and olfactory marker protein (OMP) from the biological sample; and comparing the levels of the markers to distinguish between the normal people and mild cognitive impairment (MCI).

In addition, the present disclosure provides a method of providing information for distinguishing between normal people and Alzheimer's disease, including measuring a level of a biomarker including amyloid beta 40 (Aβ40), amyloid beta 42 (Aβ42), brain-derived neurotrophic factor (BDNF), phospho-Tau231, and olfactory marker protein (OMP) from a biological sample isolated from a subject; measuring a level of a biomarker including Tau, neuron-specific enolase (NSE), glial fibrillary acidic protein (GFAP), ubiquitin carboxy-terminal hydrolase L1 (UCH-L1), and peptidyl-prolyl isomerase (PIN1) from the biological sample; and comparing the levels of the markers to distinguish between the normal people and Alzheimer's disease.

In addition, the present disclosure provides a method of providing information for distinguishing between mild cognitive impairment (MCI) and Alzheimer's disease, including measuring a level of a biomarker including amyloid beta 40 (Aβ40), amyloid beta 42 (Aβ42), Tau, glial fibrillary acidic protein (GFAP), and phospho-Tau231 from a biological sample isolated from a subject; measuring a level of a biomarker including brain-derived neurotrophic factor (BDNF), collapsing response mediator protein-2 (CRMP2), and olfactory marker protein (OMP) from the biological sample; and comparing the levels of the markers to distinguish between mild cognitive impairment (MCI) and Alzheimer's disease.

### Effects of the Invention

According to the present disclosure, in order to distinguish between mild cognitive impairment (MCI) patients and Alzheimer's disease (AD) patients, it is possible to provide a biomarker composition that distinguishes between normal people, mild cognitive impairment (MCI) patients, and Alzheimer's disease (AD) patients by measuring levels of amyloid beta 40 (Aβ40), amyloid beta 42 (Aβ42), brain-derived neurotrophic factor (BDNF), Tau, neuron-specific enolase (NSE), glial fibrillary acidic protein (GFAP), ubiquitin carboxy-terminal hydrolase L1 (UCH-L1), peptidyl-prolyl isomerase (PIN1), collapsing response mediator protein-2 (CRMP2), phospho-Tau231, and olfactory marker protein (OMP) using biomarkers in the blood to identify correlation thereof.

### BRIEF DESCRIPTION OF THE DRAWINGS

FIG. 1 shows a result of evaluating amyloid beta 40 (Aβ40), a biomarker for diagnosis of a degenerative brain disease.

FIG. 2 shows a result of evaluating amyloid beta 42 (Aβ42), a biomarker for diagnosis of a degenerative brain disease.

FIG. 3 shows a result of evaluating brain-derived neurotrophic factor (BDNF), a biomarker for diagnosis of a degenerative brain disease.

FIG. 4 shows a result of evaluating Tau, a biomarker for diagnosis of a degenerative brain disease.

FIG. 5 shows a result of evaluating neuron-specific enolase (NSE), a biomarker for diagnosis of a degenerative brain disease.

FIG. 6 shows a result of evaluating glial fibrillary acidic protein (GFAP), a biomarker for diagnosis of a degenerative brain disease.

FIG. 7 shows a result of evaluating ubiquitin carboxy-terminal hydrolase L1 (UCH-L1), a biomarker for diagnosis of a degenerative brain disease.

FIG. 8 shows a result of evaluating peptidyl-prolyl isomerase (PIN1), a biomarker for diagnosis of a degenerative brain disease.

FIG. 9 shows a result of evaluating collapsing response mediator protein-2 (CRMP2), a biomarker for diagnosis of a degenerative brain disease.

FIG. 10 shows a result of evaluating phospho-Tau231, a biomarker for diagnosis of a degenerative brain disease.

FIG. 11 shows a result of evaluating olfactory marker protein (OMP), a biomarker for diagnosis of a degenerative brain disease.

FIG. 12 is a heatmap result for analyzing correlation of biomarkers for diagnosis of a degenerative brain disease.

### DETAILED DESCRIPTION

The terms used in the present specification have been selected from general terms that are currently widely used as much as possible while considering the functions in the present disclosure, but these may vary depending on the intention of those skilled in the art or precedents or the emergence of new technologies. Additionally, in certain cases, there are terms arbitrarily selected by the applicant, and in such cases, meanings thereof will be set forth in detail in the detailed description of the disclosure. Therefore, the terms used herein should be defined based on the meaning of the terms and the overall content of the present disclosure, rather than simply the names of the terms.

Unless otherwise defined, all terms used herein, including technical or scientific terms, have the same meaning as commonly understood by those skilled in the art to which the present disclosure pertains. Terms defined in commonly used dictionaries should be interpreted as having a meaning consistent with the meaning in the context of the relevant art, but not be interpreted in an idealized or overly formal sense, unless explicitly defined in this application.

The numerical range includes the numerical values defined in the above range. All maximum numerical limits given throughout the specification include all lower numerical limits as if the lower numerical limits were expressly written. All minimum numerical limits given throughout this specification include all higher numerical limits as if the higher numerical limits were expressly written. All numerical limits given throughout this specification will include all the better numerical range within the broader numerical range, as if the narrower numerical limits were explicitly written.

Hereinafter, the present disclosure will be described in more detail.

The present disclosure provides a diagnostic biomarker composition that distinguishes between normal people and mild cognitive impairment (MCI), including amyloid beta 40 (Aβ40), amyloid beta 42 (Aβ42), collapsing response mediator protein-2 (CRMP2), and phospho-Tau231 as active ingredients.

The diagnostic biomarker composition that distinguishes between the normal people and the mild cognitive impairment (MCI) may further include one or more selected from Tau, ubiquitin carboxy-terminal hydrolase L1 (UCH-L1), and olfactory marker protein (OMP).

In addition, the present disclosure provides a diagnostic biomarker composition that distinguishes between normal people and Alzheimer's disease, including amyloid beta 40 (Aβ40), amyloid beta 42 (Aβ42), brain-derived neurotrophic factor (BDNF), phospho-Tau231, and olfactory marker protein (OMP) as active ingredients.

The diagnostic biomarker composition that distinguishes between the normal people and Alzheimer's disease may further include one or more selected from Tau, neuron-specific enolase (NSE), glial fibrillary acidic protein (GFAP), ubiquitin carboxy-terminal hydrolase L1 (UCH-L1), and peptidyl-prolyl isomerase (PIN1).

In addition, the present disclosure provides a diagnostic biomarker composition that distinguishes between mild cognitive impairment (MCI) and Alzheimer's disease, including amyloid beta 40 (Aβ40), amyloid beta 42 (Aβ42), Tau, glial fibrillary acidic protein (GFAP), and phospho-Tau231 as active ingredients.

The diagnostic biomarker composition that distinguishes between mild cognitive impairment (MCI) and Alzheimer's disease may further include one or more selected from brain-derived neurotrophic factor (BDNF), collapsing response mediator protein-2 (CRMP2), and olfactory marker protein (OMP).

In addition, the present disclosure provides a diagnostic kit that distinguishes between normal people and mild cognitive impairment (MCI), including an agent for measuring a level of a diagnostic biomarker composition that distinguishes between the normal people and mild cognitive impairment (MCI).

In addition, the present disclosure provides a diagnostic kit that distinguishes between normal people and mild cognitive impairment (MCI), including an agent for measuring a level of a diagnostic biomarker composition that distinguishes between the normal people and Alzheimer's disease.

In addition, the present disclosure provides a diagnostic kit that distinguishes between mild cognitive impairment (MCI) and Alzheimer's disease, including an agent for measuring a level of a diagnostic biomarker composition that distinguishes between mild cognitive impairment (MCI) and Alzheimer's disease.

The agent for measuring the level of the biomarker composition is an antibody that specifically binds to each marker.

In addition, the present disclosure provides a method of providing information for distinguishing between normal people and mild cognitive impairment (MCI), including measuring a level of a biomarker consisting of amyloid beta 40 (Aβ40), amyloid beta 42 (Aβ42), collapsing response mediator protein-2 (CRMP2), and phospho-Tau231 from a biological sample isolated from a subject; measuring levels of one or more markers selected from Tau, ubiquitin carboxy-terminal hydrolase L1 (UCH-L1), and olfactory marker protein (OMP) from the biological sample; and comparing the levels of the markers to distinguish between the normal people and mild cognitive impairment (MCI).

The biological sample may be any one or more selected from blood, serum, tissue, urine, saliva, and cerebrospinal fluid.

In the biological sample, if the level of amyloid beta 40 (Aβ40) is from 5.00 pg/ml to less than 30.00 pg/ml, the level of amyloid beta 42 (Aβ42) is from 0.2 pg/ml to less than 0.88 pg/ml, the level of collapsing response mediator protein-2 (CRMP2) is from 3.00 ng/ml to less than 6.00 ng/ml, and the level of phospho-Tau231 is from 15.00 pg/ml to less than 40.00 pg/ml, it may be determined that the subject of the biological sample is likely to have mild cognitive impairment (MCI).

In the biological sample, if the level of Tau is from 3.30 pg/ml to less than 10.00 pg/ml, the level of ubiquitin carboxy-terminal hydrolase L1 (UCH-L1) is from 60.00 pg/ml to less than 90.00 pg/ml, and the level of olfactory marker protein (OMP) is greater than 0.5 ng/ml to 0.80 ng/ml or less, it may be determined that the subject of the biological sample is likely to have mild cognitive impairment (MCI).

In addition, the present disclosure provides a method of providing information for distinguishing between the normal people and Alzheimer's disease, including measuring a level of a biomarker consisting of amyloid beta 40 (Aβ40), amyloid beta 42 (Aβ42), brain-derived neurotrophic factor (BDNF), phospho-Tau231, and olfactory marker protein (OMP) from a biological sample isolated from a subject; measuring a level of a biomarker consisting of Tau, neuron-specific enolase (NSE), glial fibrillary acidic protein (GFAP), ubiquitin carboxy-terminal hydrolase L1 (UCH-L1), and peptidyl-prolyl isomerase (PIN1) from the biological sample; and comparing the levels of the markers to distinguish between normal people and Alzheimer's disease.

In the biological sample, if the level of amyloid beta 40 (Aβ40) is from 30.00 pg/ml to less than 91.00 pg/ml, the level of amyloid beta 42 (Aβ42) is less than 0.2 pg/ml, the level of brain-derived neurotrophic factor (BDNF) is from 200.00 pg/ml to less than 600.00 pg/ml, the level of phospho-Tau231 is from 40.00 pg/ml to less than 102.00 pg/ml, and the level of olfactory marker protein (OMP) is 0.50 ng/ml or less, it may be determined that the subject of the biological sample is likely to have Alzheimer's disease.

In the biological sample, if the level of Tau is from 10.00 pg/ml to less than 32.00 pg/ml, the level of neuron-specific enolase (NSE) is from 1000.00 pg/ml to less than 1400.00 pg/ml, the level of glial fibrillary acidic protein (GFAP) is from 20.00 pg/ml to less than 90.00 pg/ml, the level of ubiquitin carboxy-terminal hydrolase L1 (UCH-L1) is less than 60.00 pg/ml, and the level of peptidyl-prolyl isomerase (PIN1) is less than 4.0 pg/ml, it may be determined that the subject of the biological sample is likely to have Alzheimer's disease.

In addition, the present disclosure provides a method of providing information for distinguishing between mild cognitive impairment (MCI) and Alzheimer's disease, including measuring a level of a biomarker consisting of amyloid beta 40 (Aβ40), amyloid beta 42 (Aβ42), Tau, glial fibrillary acidic protein (GFAP), and phospho-Tau231 from a biological sample isolated from a subject; measuring a level of a biomarker consisting of brain-derived neurotrophic factor (BDNF), collapsing response mediator protein-2 (CRMP2), and olfactory marker protein (OMP) from the biological sample; and comparing the levels of the markers to distinguish between mild cognitive impairment (MCI) and Alzheimer's disease.

In the biological sample, if the level of amyloid beta 40 (Aβ40) is from 5.00 pg/ml to less than 30.00 pg/ml, the level of amyloid beta 42 (Aβ42) is 0.2 pg/ml to less than 0.88 pg/ml, the level of Tau is from 3.3 pg/ml to less than 10.00 pg/ml, the level of glial fibrillary acidic protein (GFAP) is from 6.2 pg/ml to less than 20.00 pg/ml, and the level of phospho-Tau231 is from 15.00 pg/ml to less than 40.00 pg/ml, it may be determined that the subject of the biological sample is likely to have mild cognitive impairment (MCI).

In the biological sample, if the level of amyloid beta 40 (Aβ40) is from 30.00 pg/ml to less than 91.00 pg/ml, the level of amyloid beta 42 (Aβ42) is less than 0.2 pg/ml, the level of Tau is from 10.00 pg/ml to less than 32.00 pg/ml, the level of glial fibrillary acidic protein (GFAP) is from 20.00 pg/ml to less than 90.00 pg/ml, and the level of phospho-Tau231 is from 40.00 pg/ml to less than 102.00 pg/ml, it may be determined that the subject of the biological sample is likely to have Alzheimer's disease.

In the biological sample, if the level of brain-derived neurotrophic factor (BDNF) is from 600.00 pg/ml to less than 900.00 pg/ml, the level of collapsing response mediator protein-2 (CRMP2) is from 3.00 ng/ml to less than 6.00 ng/ml, and the level of olfactory marker protein (OMP) is greater than 0.5 ng/ml to 0.80 ng/ml or less, it may be determined that the subject of the biological sample is likely to have mild cognitive impairment (MCI).

In the biological sample, if the level of brain-derived neurotrophic factor (BDNF) is from 200.00 pg/ml to less than 600.00 pg/ml, the level of collapsing response mediator protein-2 (CRMP2) is from 1.00 ng/ml to less than 3.00 ng/ml, and the level of olfactory marker protein (OMP) is 0.50 ng/ml or less, it may be determined that the subject of the biological sample is likely to have Alzheimer's disease.

Hereinafter, to help the understanding of the present disclosure, experimental examples and examples will be described in detail. However, the following experimental examples and examples are merely illustrative of the content of the present disclosure, and the scope of the present disclosure is not limited to the following experimental examples and examples. The experimental examples and examples of the present disclosure are provided to more completely explain the present disclosure to those of ordinary skill in the art.

### Example

According to the present disclosure, indirect immunometric ELISA was used to measure levels of main markers and submarkers for diagnosis of a degenerative brain disease.

In order to measure the levels of main markers or submarkers, used were antibodies that specifically bind to amyloid beta 40 (Aβ40), amyloid beta 42 (Aβ42), brain-derived neurotrophic factor (BDNF), Tau, neuron-specific enolase (NSE), glial fibrillary acidic protein (GFAP), ubiquitin carboxy-terminal hydrolase L1 (UCH-L1), peptidyl-prolyl isomerase (PIN1), collapsing response mediator protein-2 (CRMP2), phospho-Tau231, and olfactory marker protein (OMP). The antibodies and experimental methods used to measure the levels of respective biomarkers were referenced from commercially available kits.

Specifically, the Human Amyloid Beta 42 ELISA Kit (Ca. ab193692) was used for amyloid beta 40 (Aβ40), the Human A042 Ultrasensitive ELISA Kit (Ca. KHB3544) for amyloid beta 42 (Aβ42), Human BDNF ELISA Kit (Ca. ab212166) for brain-derived neurotrophic factor (BDNF), Human Tau ELISA Kit (Ca. ab210972) for Tau, Human Neuron specific Enolase ELISA Kit (Ca. ab217778) for neuron-specific enolase (NSE), Human GFAP DuoSet ELISA (Ca. DY2594-05) for glial fibrillary acidic protein (GFAP), Human UCH-L1/PGP9.5 DuoSet ELISA (Ca. DY6007-05) for ubiquitin carboxy-terminal hydrolase L1 (UCH-L1), Human Protein (peptidylprolyl cis/trans isomerase) NIMA-Interacting 1 (PIN1) ELISA Kit (Ca. CSB-E11308h) for peptidyl-prolyl isomerase (PIN1), Human DPYSL2/CRMP2 ELISA Kit (Ca. LS-F37929) for collapsing response mediator protein-2 (CRMP2), Human Phosphorylated Tau 231(PTAU231) ELISA kit (Ca. MBS724296) for phospho-Tau231, and Human Olfactory Marker Protein (OMP) ELISA Kit (Ca. MBS9379411) for olfactory marker protein (OMP).

Samples to be analyzed were prepared to measure the levels of main markers or submarkers. Samples prepared were those that had been stored at 2-8° C. within 5 days from the day of collection. Using serum separator tubes (SST), samples prepared were allowed to clot for 2 hours and then centrifuged at 1000×g for 15 minutes. Plasma was collected using plasma EDTA or heparin as an anticoagulant. Within 30 minutes after collection, centrifugation was performed under condition of 1000 Xg at 2-8° C. for 15 minutes. Cell culture was used by removing particles by centrifugation at 1000 Xg, 2-8° C. for 15 minutes. Tissue homogenates were used by removing supernatants by rinsing 100 mg of tissue with 1× PBS, homogenizing in 1 ml of 1× PBS, subjecting to two freeze-thaw cycles, and centrifuging at 5000 Xg, 2-8° C. for 5 minutes.

### (1) Amyloid Beta 40 (Aβ40)

The standard sample provided in the ELISA kit or each serum sample was loaded into well by 100 μl each, and the detection antibody solution was added to every well except the blank, mixed well, and reacted at room temperature for 3 hours. After washing four times with wash buffer, the secondary antibody, anti-Rabbit IgG HRP, was loaded into all wells except the blank and reacted at room temperature for 30 minutes. After completion of the reaction, washing was performed four times using wash buffer, and stabilized chromogen was treated, followed by reaction at room temperature for 30 minutes with light blocked. After checking that the chromoreaction of HRP-substrates was induced, the reaction was stopped by adding a stop solution, and the absorbance was measured at 450 nm.

### (2) Amyloid Beta 42 (Aβ42)

The standard sample provided in the ELISA kit or each serum sample was loaded into the well by 100 μl each, and the detection antibody solution was added to all wells except the blank, mixed well, and reacted at room temperature for 3 hours or at 40° C. overnight (O/N). After washing four times with wash buffer, the secondary antibody solution was loaded into all wells except the blank and reacted at room temperature for 30 minutes. After completion of the reaction, washing was performed four times using wash buffer, and stabilized chromogen was treated, followed by reaction at room temperature for 30 minutes with light blocked. After checking that the chromoreaction of HRP-substrates was induced, the reaction was stopped by adding a stop solution, and the absorbance was measured at 450 nm.

### (3) Brain-Derived Neurotrophic Factor (BDNF)

The standard sample provided in the ELISA kit or each serum sample was loaded into the well by 100 μl each, and antibody cocktail was added to each well, followed by reaction while shaking at 400 rpm for 1 hour at room temperature. After completion of the reaction, washing was performed three times using wash buffer, and TMB development solution was treated, followed by reaction while shaking at 400 rpm at room temperature for 30 minutes with light blocked. After checking that the chromoreaction of HRP-substrates was induced, the reaction was stopped by adding a stop solution, and the absorbance was measured at 450 nm.

### (4) Tau

The standard sample provided in the ELISA kit or each serum sample was loaded into the well by 100 μl each, and antibody cocktail was added to each well, followed by reaction while shaking at 400 rpm for 1 hour at room temperature. After completion of the reaction, washing was performed three times using wash buffer, and TMB development solution was treated, followed by reaction while shaking at 400 rpm at room temperature for 30 minutes with light blocked. After checking that the chromoreaction of HRP-substrates was induced, the reaction was stopped by adding a stop solution, and the absorbance was measured at 450 nm.

### (5) Neuron-Specific Enolase (NSE)

The standard sample provided in the ELISA kit or each serum sample was loaded into the well by 100 μl each, and antibody cocktail was added to each well, followed by reaction while shaking at 400 rpm for 1 hour at room temperature. After completion of the reaction, washing was performed three times using wash buffer, and TMB development solution was treated, followed by reaction while shaking at 400 rpm at room temperature for 30 minutes with light blocked. After checking that the chromoreaction of HRP-substrates was induced, the reaction was stopped by adding a stop solution, and the absorbance was measured at 450 nm.

### (6) Glial Fibrillary Acidic Protein (GFAP)

The capture antibody was loaded into a 96-well plate and reacted overnight (O/N) at room temperature to immobilize the capture antibody on the well surface. After washing three times, the reagent diluent was loaded into each well, and the blocking was carried out by reacting at room temperature for more than 1 hour. After completion of the reaction, washing was performed three times with wash buffer. The standard sample provided in the ELISA kit or each serum sample was loaded into the well by 100 μl each and reacted at room temperature for 2 hours. After washing three times with wash buffer, biotinylated detection antibody was loaded into each well and reacted at room temperature for 2 hours. After washing three times with wash buffer, streptavidin-HRP was loaded into each well and reacted at room temperature for 20 minutes with light blocked. After completion of the reaction, washing was performed three times using wash buffer, and then the substrate solution was added and reacted at room temperature for 30 minutes with light blocked. After checking that the chromoreaction of HRP-substrates was induced, the reaction was stopped by adding a stop solution, and the absorbance was measured at 450 nm.

### (7) Ubiquitin Carboxy-Terminal Hydrolase L1 (UCH-L1)

The capture antibody was loaded into a 96-well plate and reacted overnight (O/N) at room temperature to immobilize the capture antibody on the well surface. After washing three times, the reagent diluent was loaded into each well, and the blocking was performed by reacting at room temperature for more than 1 hour. After completion of the reaction, washing was performed three times with wash buffer. The standard sample provided in the ELISA kit or each serum sample was loaded into the well by 100 μl each and reacted at room temperature for 2 hours. After washing three times with wash buffer, biotinylated detection antibody was loaded into each well and reacted at room temperature for 2 hours. After washing three times with wash buffer, streptavidin-HRP was loaded into each well and reacted at room temperature for 20 minutes with light blocked. After completion of the reaction, washing was performed three times using wash buffer, and then the substrate solution was added and reacted at room temperature for 30 minutes with light blocked. After checking that the chromoreaction of HRP-substrates was induced, the reaction was stopped by adding a stop solution, and the absorbance was measured at 450 nm.

### (8) Peptidyl-Prolyl Isomerase (PIN1)

The standard sample provided in the ELISA kit or each serum sample was loaded into the well by 100 μl each and reacted at 37° C. for 2 hours. After removing the reaction solution, 1× biotinylated detection antibody was loaded into each well and reacted at 37° C. After washing three times with wash buffer after completion of the reaction, 1× HRP-avidin was loaded into each well and reacted at 37° C. for 1 hour. After washing five times with wash buffer, the TMB substrate was treated and reacted at 37° C. for 30 minutes with light blocked. After checking that the chromoreaction of HRP-substrates was induced, the reaction was stopped by adding a stop solution, and the absorbance was measured at 450 nm.

### (9) Collapsing Response Mediator Protein-2 (CRMP2)

The standard sample provided in the ELISA kit or each serum sample was loaded into the well by 100 μl each and reacted at 37° C. for 2 hours. After removing the reaction solution, Detection reagent A was loaded into each well and reacted at 37° C. for an hour. After removing the reaction solution, washing was performed three times using wash buffer, and then Detection reagent B was loaded into each well, followed by reaction at 37° C. for 1 hour. After washing five times with wash buffer, the TMB substrate was added and reacted at 37° C. for 30 minutes with light blocked. After checking that the chromoreaction of HRP-substrates was induced, the reaction was stopped by adding a stop solution, and the absorbance was measured at 450 nm.

### (10) Phospho-Tau231

The standard sample provided in the ELISA kit or each serum sample was loaded into the well by 100 μl each, and the enzyme conjugate was treated in every well except the blank, followed by reaction at 37° C. for 1 hour. After washing all wells including blank wells five times using wash buffer, Substrate A and Substrate B were sequentially added to every well and reacted at 37° C. for 30 minutes with light blocked. After checking that the chromoreaction of HRP-substrates was induced, the reaction was stopped by adding a stop solution, and the absorbance was measured at 450 nm.

### (11) Olfactory Marker Protein (OMP)

The standard sample provided in the ELISA kit or each serum sample was loaded into the well by 100 μl each, and HRP-conjugate reagent was treated in every well except the blank, followed by reaction at 37° C. for 1 hour. After washing all wells including blank wells four times using wash buffer after completion of the reaction, Chromogen Solution A and B were sequentially treated to all wells and reacted at 37° C. for 30 minutes with light blocked. After checking that the chromoreaction of HRP-substrates was induced, the reaction was stopped by adding a stop solution, and the absorbance was measured at 450 nm.

The concentration of each marker protein in all samples was calculated using the standard curves that were obtained from the corresponding experiments.

### Experimental Example

### (1) Comparison of Expression Levels of Main Markers and Submarkers

To identify main markers and submarkers for diagnosing a degenerative brain disease according to the present disclosure, 65 serum samples were provided from normal volunteers, mild cognitive impairment (MCI) patients, and Alzheimer's disease (AD) patients recruited from Dx Biosamples, LLC, a human biospecimen contract procurement organization, and the levels of main markers and submarkers present in each serum were measured.

The mild cognitive impairment (MCI) patients and the Alzheimer's disease (AD) patients are those diagnosed via a diagnostic method that is subdivided into seven-stage grade for Alzheimer's diagnosis. The seven-stage grade for diagnosis of Alzheimer's disease is as follows.

AD Stage 1: Cognitive function appears normal, but pathological changes appear in the brain.

AD Stage 2: Mild cognitive impairment. Noticing problems with memory, such as forgetting familiar words, names, or locations of objects. Pathological changes appear in the brain.

AD Stage 3: Mild cognitive impairment (MCI). Difficulty remembering words or names. Getting lost or not finding right words. Losing or misplacing valuables. Reading sentences but barely remembering. Pathological changes appear in the brain.

AD Stage 4: Moderate cognitive decline. Early-stage Alzheimer's disease (Stage 1). Short-term memory declines and parts of the past day are not remembered. Difficulty performing complex tasks.

AD Stage 5: Severe cognitive decline. Intermediate-stage Alzheimer's (Stage 2). Cognitive function continues to decline, and assistance from others is needed for daily life. Failing to remember important information at the interview such as current address, phone number, and the name of university graduated. Confused about the current location, date, day of the week, or season. Inability to do easy mental calculations.

AD Stage 6: Very severe cognitive decline. Severe Alzheimer's disease (Stage 3). Memory problems worsen with marked personality changes. Requiring a lot of help with normal daily activities. Remembering one's own name, but not the names or faces of people around. Disruption of sleep/wake cycles. Frequently experiencing urinary and fecal incontinence. Suspicion, forgetfulness, hallucinations, or obsessive symptoms appear.

AD Stage 7: End-stage Alzheimer's. A stage nearing death, with a motor function declining and communication becoming impossible.

Using the method of the above Examples, the levels of amyloid beta 40 (Aβ40), amyloid beta 42 (Aβ42), brain-derived neurotrophic factor (BDNF), Tau, neuron-specific enolase (NSE), glial fibrillary acidic protein (GFAP), ubiquitin carboxy-terminal hydrolase L1 (UCH-L1), peptidyl-prolyl isomerase (PIN1), collapsing response mediator protein-2 (CRMP2), phospho-Tau231, or olfactory marker protein (OMP) were measured from blood samples of each classified control and experimental group, and the expression levels of each marker were compared using a bar graph.

TABLE 1

| Biomarker | Normal | MCI | AD [symbol] |
|---|---|---|---|
| Aβ40 | 3.54 ± 0.41 | 10.23 ± 4.69 | 57.86 ± 22.49 |
| (pg/ml) | | | |
| Aβ42 | 1.22 ± 0.31 | 0.55 ± 0.31 | 0.04 ± 0.03 |
| (pg/ml) | | | |
| BDNF | 969.19 ± 53.59 | 763.53 ± 106.25 | 406.55 ± 136.81 |
| (pg/ml) | | | |
| Tau (pg/ml) | 2.63 ± 0.44 | 4.85 ± 1.47 | 23.25 ± 8.61 |
| NSE (pg/ml) | 730.00 ± 84.65 | 973.48 ± 106.00 | 1204.59 ± 184.54 |
| GFAP | 5.56 ± 0.68 | 7.30 ± 0.92 | 55.06 ± 33.32 |
| (pg/ml) | | | |
| UCH-L1 | 107.90 ± 9.39 | 77.32 ± 10.89 | 45.38 ± 13.27 |
| (pg/ml) | | | |
| PIN1 | 5.83 ± 0.43 | 4.85 ± 0.47 | 3.10 ± 0.41 |
| (pg/ml) | | | |
| CRMP2 | 1.19 ± 0.11 | 4.86 ± 0.79 | 2.00 ± 0.50 |
| (ng/ml) | | | |
| p-Tau231 | 2.24 ± 0.45 | 28.15 ± 8.86 | 74.58 ± 26.80 |
| (pg/ml) | | | |
| OMP | 1.11 ± 0.24 | 0.70 ± 0.27 | 0.47 ± 0.22 |
| (ng/ml) | | | |

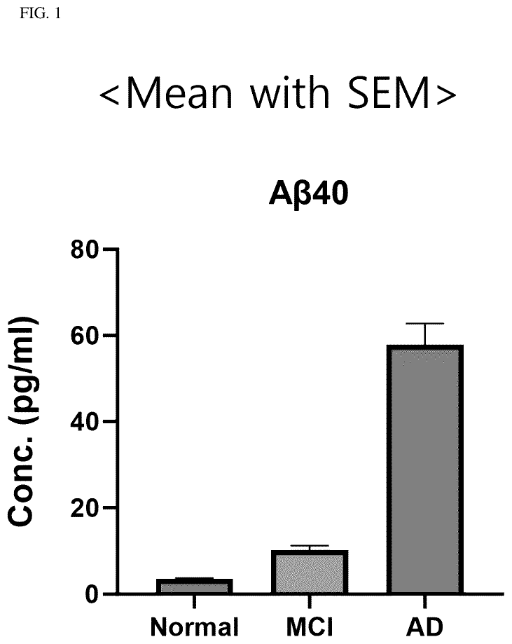

As shown in Table 1 and FIGS. 1 to 11, there were significant differences in the expression levels of amyloid beta 40 (Aβ40), amyloid beta 42 (Aβ42), brain-derived neurotrophic factor (BDNF), Tau, neuron-specific enolase (NSE), glial fibrillary acidic protein (GFAP), ubiquitin carboxy-terminal hydrolase L1 (UCH-L1), peptidyl-prolyl isomerase (PIN1), collapsing response mediator protein-2 (CRMP2), phospho-Tau231, and olfactory marker protein (OMP) in the sera of normal volunteers, mild cognitive impairment (MCI) patients, and Alzheimer's disease (AD) patients.

As shown in FIG. 1, the level of amyloid beta 40 (Aβ40) was shown to be in the level of 3.54±0.41 pg/ml in the serum of normal volunteers, 10.23±4.69 pg/ml in the serum of mild cognitive impairment (MCI) patients, and 57.86±22.49 pg/ml in the serum of Alzheimer's disease (AD) patients.

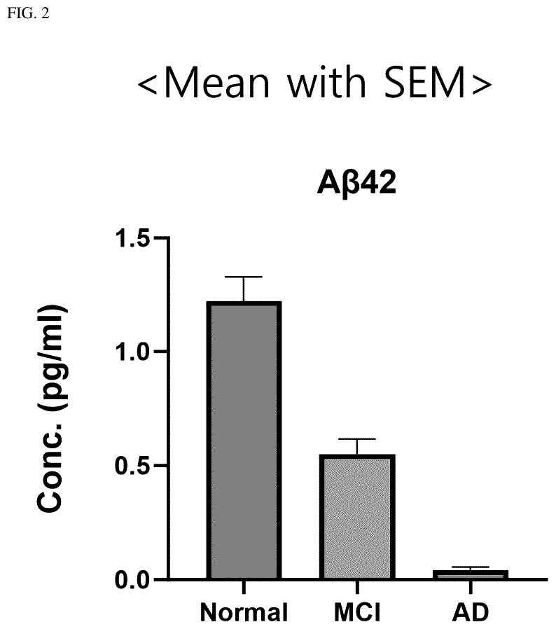

As shown in FIG. 2, the level of amyloid beta 42 (Aβ42) was shown to be in the level of 1.22±0.31 pg/ml in the serum of normal volunteers, 0.55±0.31 pg/ml in the serum of mild cognitive impairment (MCI) patients, and 0.04±0.03 pg/ml in the serum of Alzheimer's disease (AD) patients.

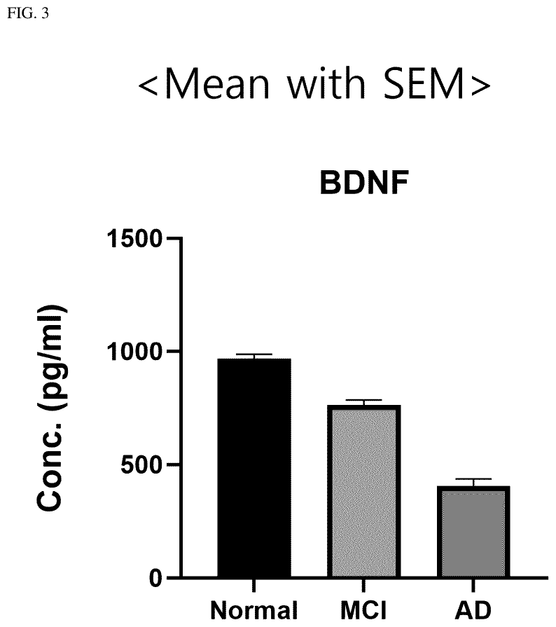

As shown in FIG. 3, the level of brain-derived neurotrophic factor (BDNF) was shown to be in the level of 969.19±53.59 pg/ml in the serum of normal volunteers, 763.53±106.25 pg/ml in the serum of mild cognitive impairment (MCI) patients, and 406.55±136.81 pg/ml in the serum of Alzheimer's disease (AD) patients.

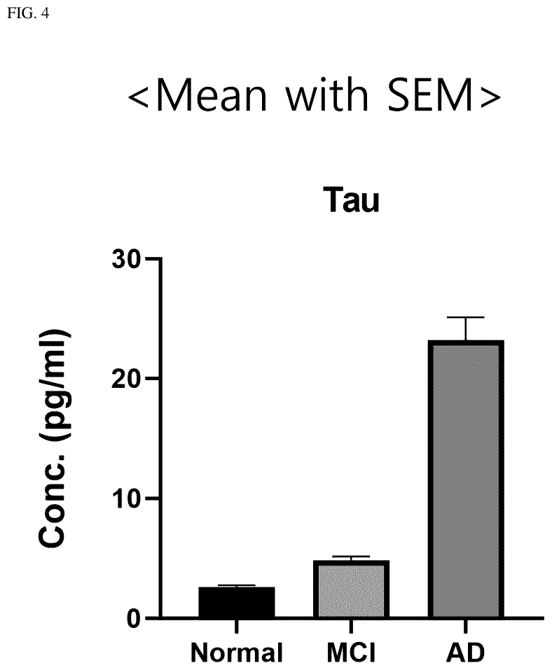

As shown in FIG. 4, the level of Tau was shown to be in the level of 2.63±0.44 pg/ml in the serum of normal volunteers, 4.85±1.47 pg/ml in the serum of mild cognitive impairment (MCI) patients, and 23.25±8.61 pg/ml in the serum of Alzheimer's disease (AD) patients.

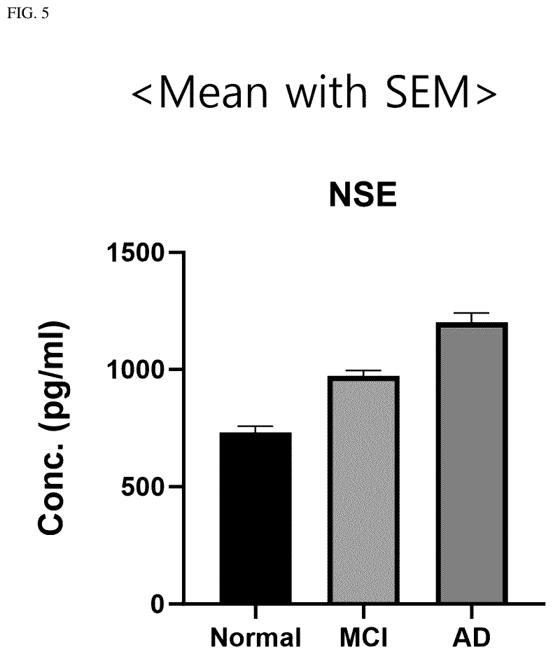

As shown in FIG. 5, the level of neuron-specific enolase (NSE) was shown to be in the level of 730.00±84.65 pg/ml in the serum of normal volunteers, 973.48±106.00 pg/ml in the serum of mild cognitive impairment (MCI) patients, and 1204.59±184.54 pg/ml in the serum of Alzheimer's disease (AD) patients.

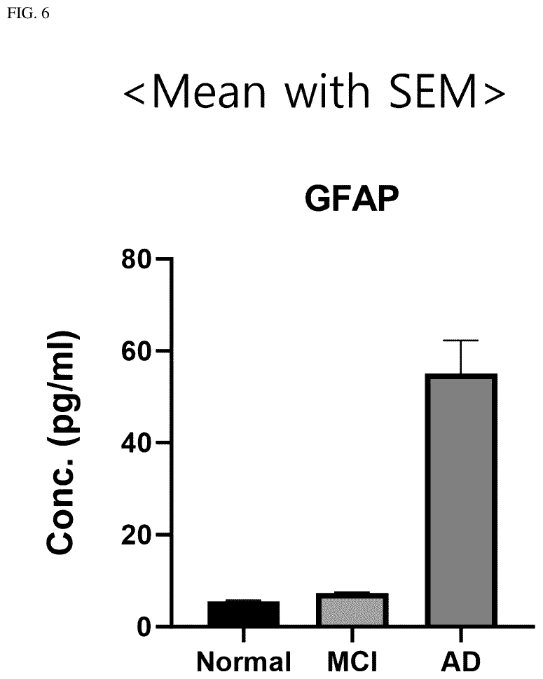

As shown in FIG. 6, the level of glial fibrillary acidic protein (GFAP) was shown to be in the level of 5.56±0.68 pg/ml in the serum of normal volunteers, 7.30±0.92 pg/ml in the serum of mild cognitive impairment (MCI) patients, and 55.06±33.32 pg/ml in the serum of Alzheimer's disease (AD) patients.

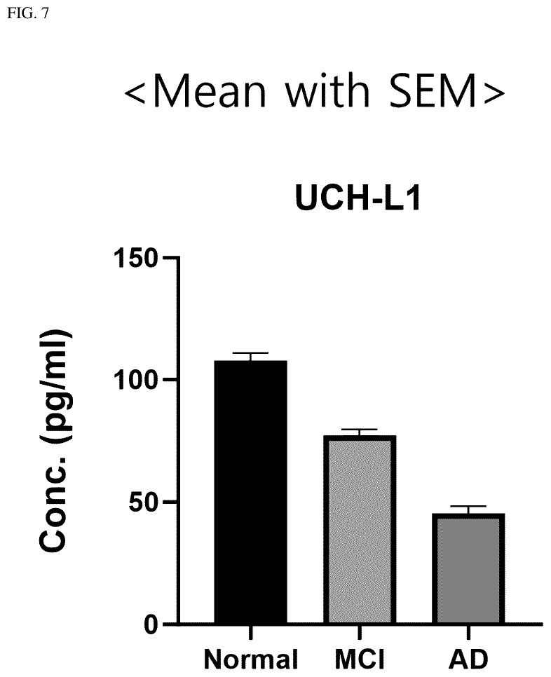

As shown in FIG. 7, the level of ubiquitin carboxy-terminal hydrolase L1 (UCH-L1) was shown to be in the level of 107.90±9.39 pg/ml in the serum of normal volunteers, 77.32±10.89 pg/ml in the serum of mild cognitive impairment (MCI) patients, and 45.38±13.27 pg/ml in the serum of Alzheimer's disease (AD) patients.

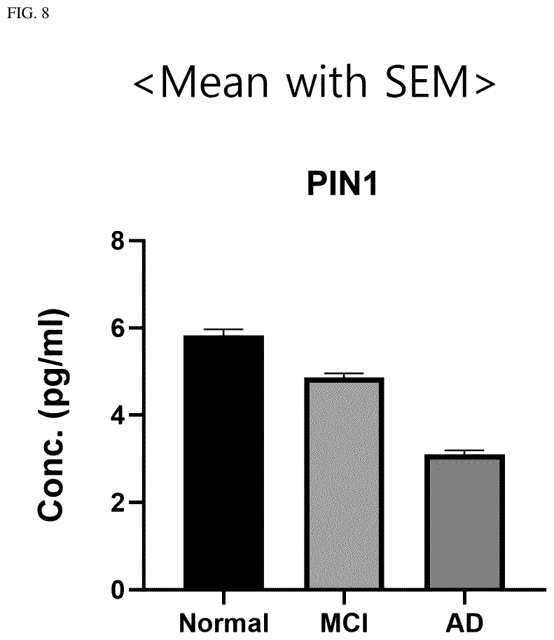

As shown in FIG. 8, the level of peptidyl-prolyl isomerase (PIN1) was shown to be in the level of 5.83±0.43 pg/ml in the serum of normal volunteers, 4.85±0.47 pg/ml in the serum of mild cognitive impairment (MCI) patients, and 3.10±0.41 pg/ml in the serum of Alzheimer's disease (AD) patients.

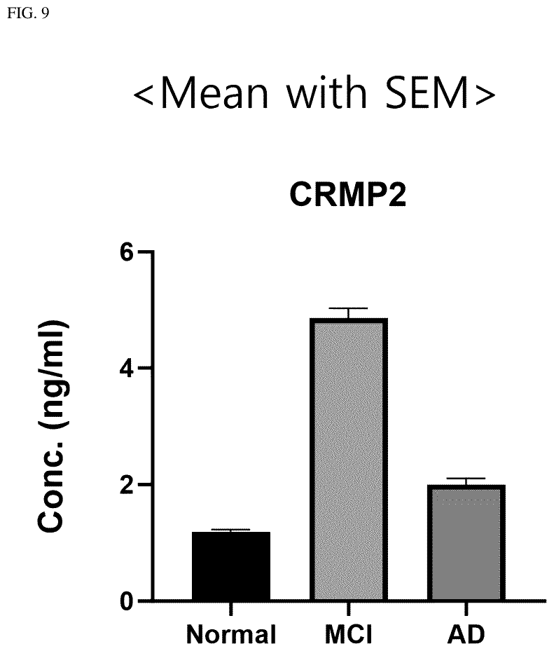

As shown in FIG. 9, the level of collapsing response mediator protein-2 (CRMP2) was shown to be in the level of 1.19±0.11 pg/ml in the serum of normal volunteers, 4.86±0.79 pg/ml in the serum of mild cognitive impairment (MCI) patients, and 2.00±0.50 pg/ml in the serum of Alzheimer's disease (AD) patients.

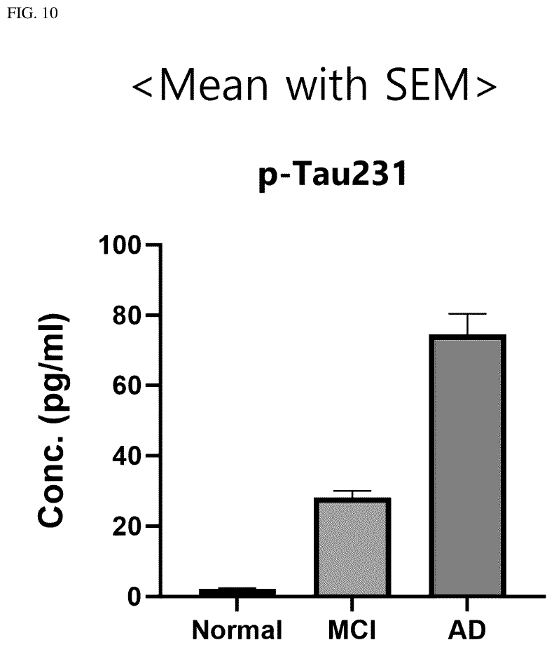

As shown in FIG. 10, the level of phospho-Tau231 was shown to be in the level of 2.24±0.45 pg/ml in the serum of normal volunteers, 28.15±8.86 pg/ml in the serum of mild cognitive impairment (MCI) patients, and 74.58±26.80 pg/ml in the serum of Alzheimer's disease (AD) patients.

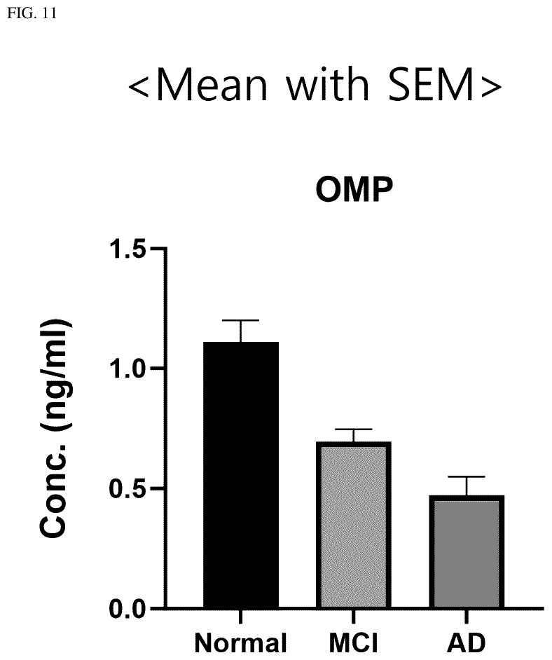

As shown in FIG. 11, the level of olfactory marker protein (OMP) was shown to be in the level of 1.11±0.24 pg/ml in the serum of normal volunteers, 0.70±0.27 pg/ml in the serum of mild cognitive impairment (MCI) patients, and 0.47±0.22 pg/ml in the serum of Alzheimer's disease (AD) patients.

### (2) Analysis of Correlation Between Main Markers and Submarkers

The expression levels of Aβ40, Aβ42, BDNF, Tau, NSE, GFAP, UCH-L1, Pin1, CRMP2, p-Tau231, and OMP were quantified in the serum of normal volunteers, mild cognitive impairment (MCI) patients, and Alzheimer's disease (AD) patients, and the average of each quantified value was calculated. Here, when the lowest average value of each experimental group for each marker was set to 1, the ratio of the result values of the remaining experimental groups was calculated. In the same way, the results were calculated for a total of 11 markers, followed by creation of a heat map using those values.

For Aβ40, the average value for expression level of Aβ40 in the serum of the Normal experimental group was set to 1, and the ratio of the average value for expression level of Aβ40 in the serum of the MCI and AD experimental groups was calculated. For Aβ42, the average value for expression level of Aβ42 in the serum of the AD experimental group was set to 1, and the ratio of the average value for expression level of Aβ42 in the serum of the Normal and MCI experimental groups was calculated. For BDNF, the average value for expression level of BDNF in the serum of the AD experimental group was set to 1, and the ratio of the average value for expression level of BDNF in the serum of the Normal and MCI experimental groups was calculated. For Tau, the average value for expression level of Tau in the serum of the Normal experimental group was set to 1, and the ratio of the average value for expression level of Tau in the serum of the MCI and AD experimental groups was calculated. For NSE, the average value for expression level of NSE in the serum of the Normal experimental group was set to 1, and the ratio of the average value for expression level of NSE in the serum of the MCI and AD experimental groups was calculated. For GFAP, the average value for expression level of GFAP in the serum of the Normal experimental group was set to 1, and the ratio of the average value for expression level of GFAP in the serum of the MCI and AD experimental groups was calculated. For UCH-L1, the average value for expression level of UCH-L1 in the serum of the AD experimental group was set to 1, and the ratio of the average value for expression level of UCH-L1 in the serum of the Normal and MCI experimental groups was calculated. For Pin1, the average value for expression level of Pin1 in the serum of the AD experimental group was set to 1, and the ratio of the average value for expression level of Pin1 in the serum of the Normal and MCI experimental groups was calculated. For CRMP2, the average value for expression level of CRMP2 in the serum of the Normal experimental group was set to 1, and the ratio of the average value for expression level of CRMP2 in the serum of the MCI and AD experimental groups was calculated. For phospho-Tau231 (p-Tau231), the average value for expression level of p-Tau231 in the serum of the Normal experimental group was set to 1, and the ratio of the average value for expression level of p-Tau231 in the serum of the MCI and AD experimental groups was calculated. For OMP, the average value for expression level of OMP in the serum of the AD experimental group was set to 1, and the ratio of the average value for expression level of OMP in the serum of the Normal and MCI experimental groups was calculated.

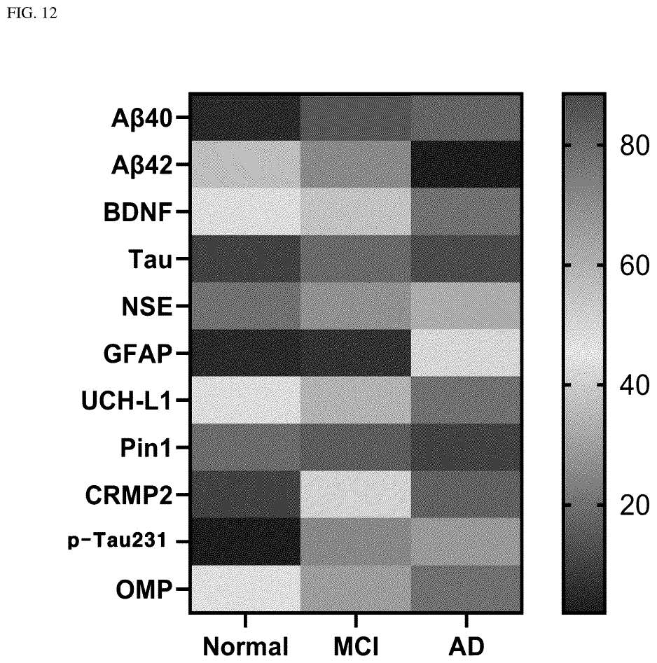

By comparing the differences in expression levels shown in FIG. 12, biomarkers that distinguish between normal volunteers (Normal), mild cognitive impairment (MCI) patients, and Alzheimer's disease (AD) patients were selected.

Comparing normal volunteers and patients with mild cognitive impairment (MCI), BDNF, NSE, GFAP, and Pin1 showing similar tendency among 11 biomarkers were excluded. Of the remaining 8 markers, significant differences in expression levels were observed in amyloid beta 40 (Aβ40), amyloid beta 42 (Aβ42), collapsing response mediator protein-2 (CRMP2), and phospho-Tau231, as well as in Tau, ubiquitin carboxy-terminal hydrolase L1 (UCH-L1), and olfactory marker protein (OMP).

Comparing normal volunteers (Normal) and Alzheimer's disease (AD) patients, CRMP2 showing a similar tendency among 11 biomarkers was excluded. Of the remaining 10 markers, significant differences in expression levels were observed in amyloid beta 40 (Aβ40), amyloid beta 42 (Aβ42), phospho-Tau231, brain-derived neurotrophic factor (BDNF), and olfactory marker protein (OMP), as well as in Tau, neuron-specific enolase (NSE), glial fibrillary acidic protein (GFAP), ubiquitin carboxy-terminal hydrolase L1 (UCH-L1), and peptidyl-prolyl isomerase (PIN1).

Comparing patients with mild cognitive impairment (MCI) and Alzheimer's disease (AD), NSE, UCH-L1, and Pin1 showing similar tendency among 11 biomarkers were excluded. Of the remaining 8 markers, significant differences in expression levels were observed in amyloid beta 40 (Aβ40), amyloid beta 42 (Aβ42), Tau, glial fibrillary acidic protein (GFAP), and phospho-Tau231, as well as in brain-derived neurotrophic factor (BDNF), collapsing response mediator protein-2 (CRMP2), and olfactory marker protein (OMP).

Through the heatmap results, biomarkers that distinguish between normal volunteers (Normal), mild cognitive impairment (MCI) patients, and Alzheimer's disease (AD) patients were classified as shown in Table 2 below, proving that the normal volunteers (Normal), mild cognitive impairment (MCI) patients, and Alzheimer's disease (AD) patients can be distinguished using the following biomarkers.

TABLE 2

| Classification | Normal vs MCI | Normal vs AD | MCI vs AD |
|---|---|---|---|
| Main marker | Aβ40, Aβ42, | Aβ40, Aβ42, | Aβ40, Aβ42, Tau, |
| | CRMP2, p-Tau | BDNF, p-Tau, | GFAP, p-Tau |
| | | OMP | |
| Submarker | Tau, UCH-L1, | Tau, NSE, GFAP, | BDNF, CRMP2, |
| | OMP | UCH-L1, Pin1 | OMP |

As described above, since a specific part of the present invention is described in detail, for those skilled in the art, it is clear that the specific description is merely preferred embodiments and the scope of the present disclosure is not limited thereby. Thus, the substantial scope of the present disclosure will be defined by the appended claims and their equivalents.

What is claimed is:

1. A method of providing information for distinguishing between normal people and mild cognitive impairment (MCI), the method comprising:
measuring a level of a biomarker comprising amyloid beta 40 (Aβ40), amyloid beta 42 (Aβ42), collapsing response mediator protein-2 (CRMP2), and phospho-Tau231 from a biological sample isolated from a subject;
measuring levels of one or more markers selected from Tau, ubiquitin carboxy-terminal hydrolase L1 (UCH-L1), and olfactory marker protein (OMP) from the biological sample; and
comparing the levels of the markers to distinguish between the normal people and mild cognitive impairment (MCI).

2. The method of claim 1, wherein, in the biological sample,
if the level of amyloid beta 40 (Aβ40) is from 5.00 pg/ml to less than 30.00 pg/ml,
the level of amyloid beta 42 (Aβ42) is from 0.2 pg/ml to less than 0.88 pg/ml,
the level of collapsing response mediator protein-2 (CRMP2) is from 3.00 ng/ml to less than 6.00 ng/ml, and
the level of phospho-Tau231 is from 15.00 pg/ml to less than 40.00 pg/ml,
it is determined that the subject of the biological sample is likely to have mild cognitive impairment (MCI).

3. The method of claim 1, wherein, in the biological sample,
if the level of Tau is from 3.30 pg/ml to less than 10.00 pg/ml,
the level of ubiquitin carboxy-terminal hydrolase L1 (UCH-L1) is from 60.00 pg/ml to less than 90.00 pg/ml, and
the level of olfactory marker protein (OMP) is greater than 0.5 ng/ml to 0.80 ng/ml or less,
it is determined that the subject of the biological sample is likely to have mild cognitive impairment (MCI).

4. A method of providing information for distinguishing between normal people and Alzheimer's disease, the method comprising:
measuring a level of a biomarker comprising amyloid beta 40 (Aβ40), amyloid beta 42 (Aβ42), brain-derived neurotrophic factor (BDNF), phospho-Tau231, and olfactory marker protein (OMP) from a biological sample isolated from a subject;
measuring a level of a biomarker comprising Tau, neuron-specific enolase (NSE), glial fibrillary acidic protein (GFAP), ubiquitin carboxy-terminal hydrolase L1 (UCH-L1), and peptidyl-prolyl isomerase (PIN1) from the biological sample; and
comparing the levels of the markers to distinguish between the normal people and Alzheimer's disease.

5. The method of claim 4, wherein, in the biological sample,
if the level of amyloid beta 40 (Aβ40) is from 30.00 pg/ml to less than 91.00 pg/ml,
the level of amyloid beta 42 (Aβ42) is less than 0.2 pg/ml,
the level of brain-derived neurotrophic factor (BDNF) is from 200.00 pg/ml to less than 600.00 pg/ml,
the level of phospho-Tau231 is from 40.00 pg/ml to less than 102.00 pg/ml, and the level of olfactory marker protein (OMP) is 0.50 ng/ml or less,
it is determined that the subject of the biological sample is likely to have Alzheimer's disease.

6. The method of claim 4, wherein, in the biological sample,
if the level of Tau is from 10.00 pg/ml to less than 32.00 pg/ml,
the level of neuron-specific enolase (NSE) is from 1000.00 pg/ml to less than 1400.00 pg/ml,
the level of glial fibrillary acidic protein (GFAP) is from 20.00 pg/ml to less than 90.00 pg/ml,
the level of ubiquitin carboxy-terminal hydrolase L1 (UCH-L1) is less than 60.00 pg/ml, and
the level of peptidyl-prolyl isomerase (PIN1) is less than 4.0 pg/ml,
it is determined that the subject of the biological sample is likely to have Alzheimer's disease.

7. A method of providing information for distinguishing between mild cognitive impairment (MCI) and Alzheimer's disease, the method comprising:
measuring a level of a biomarker comprising amyloid beta 40 (Aβ40), amyloid beta 42 (Aβ42), Tau, glial fibrillary acidic protein (GFAP), and phospho-Tau231 from a biological sample isolated from a subject;
measuring a level of a biomarker comprising brain-derived neurotrophic factor (BDNF), collapsing response mediator protein-2 (CRMP2), and olfactory marker protein (OMP) from the biological sample; and
comparing the levels of the markers to distinguish between mild cognitive impairment (MCI) and Alzheimer's disease.

8. The method of claim 7, wherein, in the biological sample,
if the level of amyloid beta 40 (Aβ40) is from 5.00 pg/ml to less than 30.00 pg/ml,
the level of amyloid beta 42 (Aβ42) is 0.2 pg/ml to less than 0.88 pg/ml,
the level of Tau is from 3.3 pg/ml to less than 10.00 pg/ml,
the level of glial fibrillary acidic protein (GFAP) is from 6.2 pg/ml to less than 20.00 pg/ml, and
the level of phospho-Tau231 is from 15.00 pg/ml to less than 40.00 pg/ml,
it is determined that the subject of the biological sample is likely to have mild cognitive impairment (MCI).

9. The method of claim 7, wherein, in the biological sample,
if the level of amyloid beta 40 (Aβ40) is from 30.00 pg/ml to less than 91.00 pg/ml,
the level of amyloid beta 42 (Aβ42) is less than 0.2 pg/ml,
the level of Tau is from 10.00 pg/ml to less than 32.00 pg/ml,
the level of glial fibrillary acidic protein (GFAP) is from 20.00 pg/ml to less than 90.00 pg/ml, and
the level of phospho-Tau231 is from 40.00 pg/ml to less than 102.00 pg/ml,
it is determined that the subject of the biological sample is likely to have Alzheimer's disease.

10. The method of claim 7, wherein, in the biological sample,
if the level of brain-derived neurotrophic factor (BDNF) is from 600.00 pg/ml to less than 900.00 pg/ml,
the level of collapsing response mediator protein-2 (CRMP2) is from 3.00 ng/ml to less than 6.00 ng/ml, and
the level of olfactory marker protein (OMP) is greater than 0.5 ng/ml to 0.80 ng/ml or less,
it is determined that the subject of the biological sample is likely to have mild cognitive impairment (MCI).

11. The method of claim 7, wherein, in the biological sample,
if the level of brain-derived neurotrophic factor (BDNF) is from 200.00 pg/ml to less than 600.00 pg/ml,
the level of collapsing response mediator protein-2 (CRMP2) is from 1.00 ng/ml to less than 3.00 ng/ml, and
the level of olfactory marker protein (OMP) is 0.50 ng/ml or less,
it is determined that the subject of the biological sample is likely to have Alzheimer's disease.
